(19) United States
(12) Patent Application Publication
(10) Pub. No.: US 2024/0197482 A1
(43) Pub. Date: Jun. 20, 2024

(54) ACETABULAR CUP WITH REGIONS OF DIFFERING FLEXIBILITY

(71) Applicant: ADLER ORTHO S.P.A., CORMANO (IT)

(72) Inventors: Jean-François GONZALEZ, TOULON (FR); Francesco TRAINA, FERRARA (IT); Duncan WHITWELL, Oxfordshire (GB); Aldo TONI, BOLOGNA (IT); Geert Hilda DE LOORE, DEURLE (BE); Georg KOESTER, BENSHEIM (DE); Koen DESMET, CASTEL GIORGIO (IT); Antonio CAMPACCI, FRAZIONE ARBIZZANO (IT); Xavier FLECHER, TRETS (FR)

(21) Appl. No.: 18/555,426

(22) PCT Filed: Dec. 13, 2021
(86) PCT No.: PCT/EP2021/085416

§ 371 Date: Oct. 13, 2023

(30) Foreign Application Priority Data
Apr. 13, 2021 (IT) 102021000009176

(51) Int. Cl. A61F 2/34; A61F 2/30; B22F 5/10; B22F 10/28; B33Y 10/00; B33Y 80/00

(52) U.S. Cl. CPC A61F 2/34; B22F 5/10; B22F 10/28; B33Y 10/00; B33Y 80/00; A61F 2002/30014; A61F 2002/3021; A61F 2002/30291; A61F 2002/30324; A61F 2002/30787; A61F 2002/30879; A61F 2002/30902; A61F 2002/30909; A61F 2002/3092; A61F 2002/3403; B22F 2301/205

(57) ABSTRACT

An acetabular cup for a hip prosthesis, which includes a cotyle body provided with a number of holes for the passage of fixing screws. T holes are connected to each other by a number of ribs which define a grid.

### CROSS-REFERENCE TO RELATED APPLICATIONS

This application is a 35 U.S.C. § 371 National Stage patent application of PCT/EP2021/085416, filed on 13 Dec. 2021, which claims the benefit of Italian patent application 102021000009176, filed on 13 Apr. 2021, the disclosures of which are incorporated herein by reference in their entirety.

### TECHNICAL FIELD

The present disclosure relates to a cotyle (or acetabular cup) with differentiated flexibility. More specifically, the disclosure relates to a cotyle that is adapted to be used in hip prostheses, both in first hip replacements and in revisions.

### BACKGROUND

As is known, in a hip prosthesis a cotyle is used, which is substantially a cup-shaped element that is inserted into the seat of the basin and the function of which is to restore the femoral joint in the seat of the basin.

As is known, the cotyle is made of titanium alloy or similar materials, and it is provided with a plurality of holes so that it can be optionally fixed with special screws into the seat defined in the basin.

Conventional cotyles are made with an elasticity, or rather rigidity, that is the same over their entire extension.

This rigidity gives rise to a phenomenon of bone density loss of the bone of the acetabulum, i.e. of the cavity that accommodates the cotyle. In substance, the cotyle has a greater rigidity than the bone that surrounds it. This entails a damage for the patient, because the bone of the acetabulum, as mentioned, deteriorates.

A cotyle must however ensure the “primary fixation”, which is obtained by the mechanical coupling of the prosthesis in the bone, optionally increased through the use of screws, and at the same time it must ensure the “secondary fixation”, obtained with the adherence of the bone into the porosity of the surface of the cotyle, i.e. osseointegration.

Obviously the rigidity of the cotyle must however be adequate so as to ensure the required mechanical performance.

### SUMMARY

The aim of the present disclosure is to provide a cotyle that has a differentiated elasticity along its surface, so as to reduce the damage to the bone surrounding the cotyle, when the cotyle is implanted in its acetabular seat.

Within this aim, the present disclosure provides a cotyle, the elasticity of which is counterbalanced by an adequate robustness to ensure the mechanical performance required by the cotyle.
the present disclosure also provides a cotyle that can be used both for a first implantation and as a revision cotyle.
the present disclosure further provides a cotyle that allows the insertion inside it of inserts made of ceramic, of chrome-cobalt alloy or of polyethylene, as in conventional cotyloid cavities.
the present disclosure also provides a cotyle that is highly reliable, easily and practically implemented and of low cost.

This aim and these and other advantages which will become better apparent hereinafter will be achieved by providing a cotyle for a hip prosthesis, which comprises a cotyle body provided with a plurality of holes for the passage of fixing screws, said holes being connected to each other by a plurality of ribs which are adapted to define a grid.

### BRIEF DESCRIPTION OF THE DRAWINGS

Further characteristics and advantages of the disclosure will become better apparent from the detailed description of a preferred, but not exclusive, embodiment of the cotyle according to the disclosure, illustrated by way of non-limiting example in the accompanying drawings wherein:

FIG. 1 is a plan view from above of the cotyle according to the present disclosure;

FIG. 2 is a side view of the cotyle according to the present disclosure;

FIG. 3 is a plan view from below of the cotyle according to the present disclosure; and

FIG. 4 is a cross-sectional view of the cotyle according to the present disclosure.

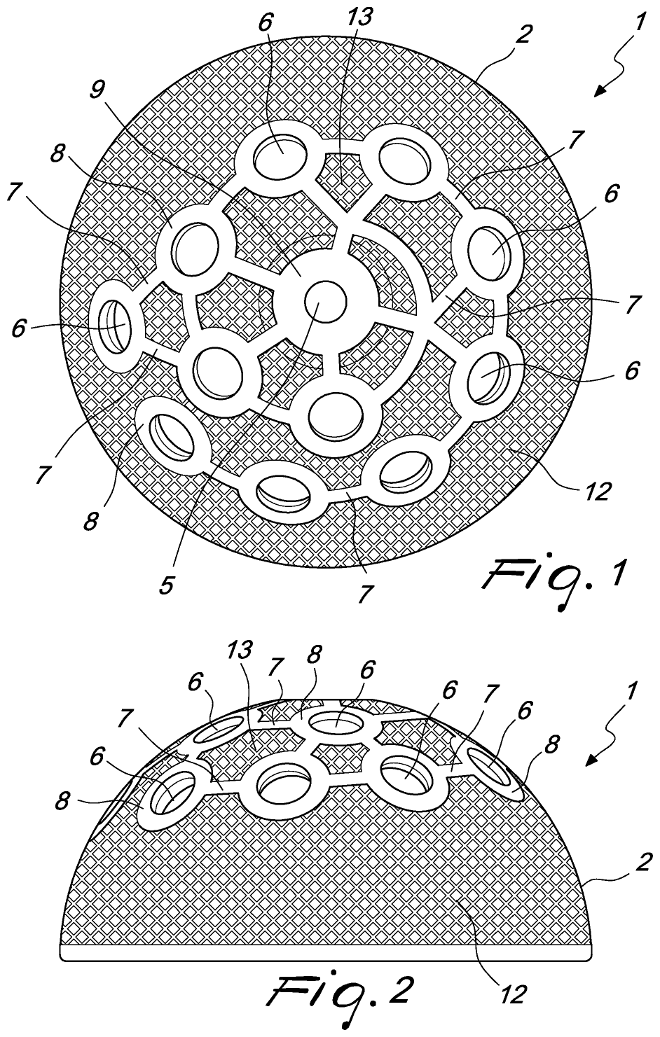

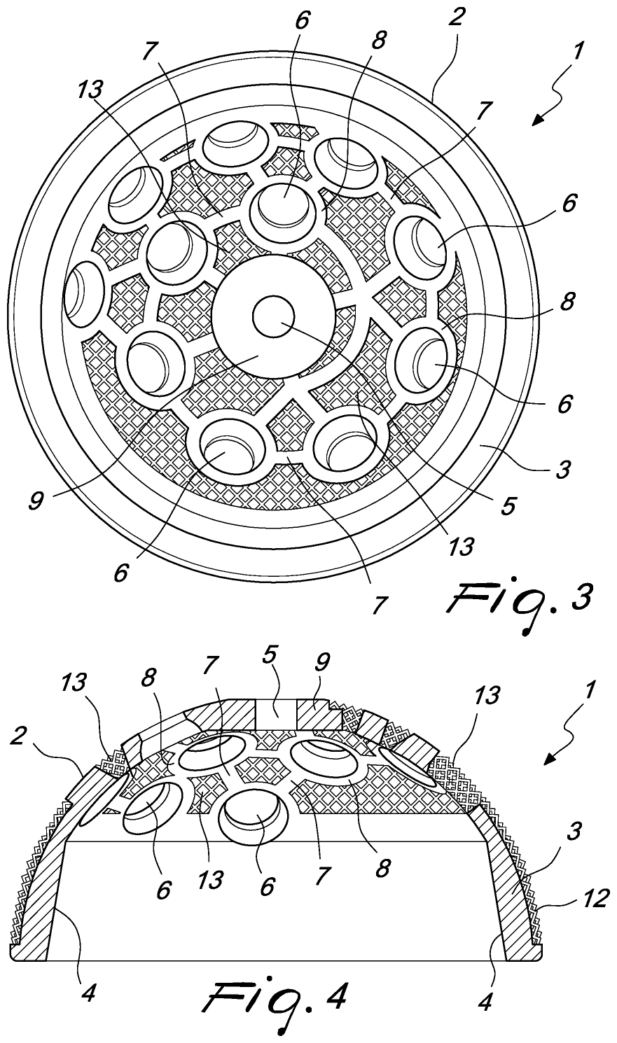

### DETAILED DESCRIPTION OF THE DRAWINGS

With reference to the figures, in which identical reference numerals designate identical elements, the cotyle according to the disclosure, generally designated by the reference numeral 1, comprises a cotyle body 2 which is characterized by an equatorial region 3 which defines internally a frustum-shaped region 4. The equatorial region 3, indicated as “base”, is characterized in that it has a solid structure, up to for example 27-46° of the meridians of the domed portion of the body 2 (the angle varies in relation to the diameter of the cotyle).

Such region 3 is covered on the external convex part by a porous mesh 12, which has for example a thickness of 1 mm.

Such region has substantially for example a height of between 17 and 18 mm (the height varies in relation to the diameter of the cotyle).

At the upper end of the cotyle is a “polar” hole 5 (necessary for fixing the cotyle to the instrument used for its fixing by way of forced mechanical coupling in the acetabulum) and a plurality of holes 6 which are arranged on the surface of the cotyle. The arrangement of the holes is obtained using a particular geometry called a “spiral”, which starts from the hole 6 closest to the polar or central hole 5 and extends, in the manner of a spiral or nautilus until it reaches the hole 6 farthest from the polar hole 5.

The holes 6 equally distributed in a spiral progression on the surface of the body 2 of the cotyle serve to allow the passage of fixing screws which may be used to fix the cotyle inside the acetabulum of the basin of the patient, if fixing by forced mechanical coupling alone is not sufficient.

The central hole 5 and the holes 6 are connected to each other by a plurality of ribs 7 which define a form of grid on the internal surface of the body 2 of the cotyle.

The ribs connect the rings 8 defined around the holes 6 of the cotyle to each other.

The rings 8 are reinforcement rings which are similar to a reinforcement ring 9 which is arranged around the central (polar) hole 5 of the cotyle.

The mesh 12 that covers the base 3 of the body 2 of the cotyle preferably has a thickness of 1 mm. Where no ribs 7 or reinforcement rings 8, 9 of holes are provided, the porous mesh 13 of different thickness has, instead, a continuity in the external convex portion.

On the external surface of the ribs 7, of the reinforcement rings 9 and of the mesh 13, pointed regions (not shown) may be provided which rise for example by 0.4 mm from the surface, while in the portions unaffected by the ribs 7 and by the reinforcement rings 8, 9 the mesh 13 has a thickness for example of 2 mm.

The cotyle is provided using the EBM (Electron Beam Melting) technology or the SLM (Selective Laser Melting) technology on titanium alloy powders.

The number of holes 6 is correlated with the diameter of the cotyle.

The cotyle thus provided has a different elasticity, from the base 3 to the polar summit of the cotyle. The base 3 is the most rigid portion and the flexibility increases in the upper region that is affected both by the holes 6 and by the ribs 7 described above.

The grid-like structure defined by the ribs 7 confers elasticity on the cotyle overall, and between the ribs 7 is the full-thickness layer (for example 2 mm) of mesh 13 (provided using the EBM (Electron Beam Melting) technique or using the SLM technique).

In practice it has been found that the cotyle according to the disclosure fully achieves the set aim and advantages, in that it makes it possible to have a flexibility such as to reduce the damage to the bone surrounding the cotyle, when the cotyle is seated in the acetabulum. Furthermore, the arrangement of the holes in a spiral makes it possible to change the seat in order to insert the fixing screws in the acetabulum simply by rotating the cotyle before its insertion by pressure coupling into the acetabular cavity.

Furthermore, the cotyle can be provided with two different types of mesh, a thinner one outside the base and a thicker one at the portions not affected by ribs and/or holes.

The cotyle, thus conceived, is susceptible of numerous modifications and variations, all of which are within the scope of the appended claims.

Moreover, all the details may be substituted by other, technically equivalent elements.

In practice, the materials used, as well as the contingent shapes and dimensions, may be any according to the requirements and to the state of the art.

1-13. (canceled)

14. A cotyle for a hip prosthesis, which comprises a cotyle body provided with a plurality of holes for the passage of fixing screws, said holes being connected to each other by a plurality of ribs which define a grid.

15. The cotyle according to claim 14, wherein said holes are surrounded by reinforcement rings.

16. The cotyle according to claim 14, further comprising a base region which is provided solid and is frustum-shaped internally.

17. The cotyle according to claim 15, wherein said ribs connect said reinforcement rings to each other.

18. The cotyle according to claim 14, further comprising a polar central hole.

19. The cotyle according to claim 18, further comprising a reinforcement ring arranged around said polar central hole.

20. The cotyle according to claim 19, wherein said base region is covered externally by a porous mesh.

21. The cotyle according to claim 19, further comprising a full-thickness porous mesh in areas of a surface of the body without ribs or reinforcement rings of the holes.

22. The cotyle according to claim 20, wherein there are pointed regions on the surface of said porous mesh that covers said base region, of said mesh, of said ribs and reinforcement rings.

23. The cotyle according to claim 14, wherein said base region has a height comprised between 12 and 14 mm.

24. The cotyle according to claim 14, wherein said holes are arranged in a configuration according to a spiral.

25. The cotyle according to claim 21, wherein said porous mesh that covers said base region is thinner than said full-thickness porous mesh that covers the areas of the surface of the body without ribs or reinforcement rings of the holes.

26. The cotyle according to claim 14, provided with EBM or SLM techniques.
